(19) United States
(12) Patent Application Publication
(10) Pub. No.: US 2011/0295080 A1
(43) Pub. Date: Dec. 1, 2011

(54) Physiology Condition Detection Device and the System Thereof

(75) Inventors: Bo Yung Chen, Jhubei City (TW); Bing Jye Kuo, Jhubei City (TW); Heng Chih Lin, Jhubei City (TW)

(73) Assignee: Ralink Technology Corporation, Jhubei City (TW)

(21) Appl. No.: 13/117,990

(22) Filed: May 27, 2011

Related U.S. Application Data
(60) Provisional application No. 61/349,868, filed on May 30, 2010.
(60) Provisional application No. 61/349,869, filed on May 30, 2010.

(51) Int. Cl. A61B 5/00

(52) U.S. Cl. 600/300

(57) ABSTRACT

A physiology condition detection device comprises a physiology condition sensor, a signal converter and an RFID processor. The physiology condition sensor is configured to sense physiology condition. The signal converter is configured to convert the sensed physiology condition to digitized physiology data. The RFID processor is configured to control the operation of the physiology condition sensor and the signal converter, and report the digitized physiology data.

### BACKGROUND OF THE INVENTION

1. Field of the Invention

The present invention relates to a physiology condition detection device and the system thereof, and more particularly, to a physiology condition detection device using radio frequency identification (RFID) technique and the system thereof.

2. Description of the Related Art

Conventionally, a patient's physiology condition, such as body temperature, electrocardiogram or brain waves, needs to be measured and monitored manually and continuously. In addition, the measured data are required to be recorded manually or stored in a computer or other devices through traditional wired communication techniques. However, many resources, such as manpower and time, are needed to perform such measuring procedure. Further, the measuring procedure may disturb the patient.

Accordingly, some research has provided methods to measure physiology condition via wireless communication techniques, such as ultra wide band (UWB) communication technique. However, most of the apparatus being used in such methods are mobile devices, which often use batteries as power sources to perform measuring procedure and report the measured results, and thus cannot support such measuring procedure continuously or for a long period of time. Therefore, there is a need to design a physiology condition detection device that is capable of measuring physiology condition wirelessly and continuously.

### SUMMARY OF THE INVENTION

The physiology condition detection device according to one embodiment of the present invention comprises a physiology condition sensor, a signal converter and an RFID processor. The physiology condition sensor is configured to sense physiology condition. The signal converter is configured to convert the sensed physiology condition to digitized physiology data. The RFID processor is configured to control the operation of the physiology condition sensor and the signal converter and report the digitized physiology data.

The physiology condition detection system according to one embodiment of the present invention comprises an RFID reader and at least one physiology condition detection device. The at least one physiology condition detection device is configured to communicate with the RFID reader. Each physiology condition detection device comprises a physiology condition sensor, a signal converter and an RFID processor. The physiology condition sensor is configured to sense physiology condition. The signal converter is configured to convert the sensed physiology condition to digitized physiology data. The RFID processor is configured to control the operation of the physiology condition sensor and the signal converter and report the digitized physiology data to the RFID reader.

The foregoing has outlined rather broadly the features and technical advantages of the present invention in order that the detailed description of the invention that follows may be better understood. Additional features and advantages of the invention will be described hereinafter, and form the subject of the claims of the invention. It should be appreciated by those skilled in the art that the conception and specific embodiment disclosed may be readily utilized as a basis for modifying or designing other structures or processes for carrying out the same purposes as those of the present invention. It should also be realized by those skilled in the art that such equivalent constructions do not depart from the spirit and scope of the invention as set forth in the appended claims.

### BRIEF DESCRIPTION OF THE DRAWINGS

The objectives and advantages of the present invention will become apparent upon reading the following description and upon referring to the accompanying drawings of which:

FIG. 1 shows a physiology condition detection device according to an embodiment of the present invention;

FIG. 2 shows the block diagram of an RFID processor according to an embodiment of the present invention;

FIG. 3 shows a physiology condition detection device according to another embodiment of the present invention; and

FIG. 4 shows a physiology condition detection system according to an embodiment of the present invention.

### DETAILED DESCRIPTION OF THE INVENTION

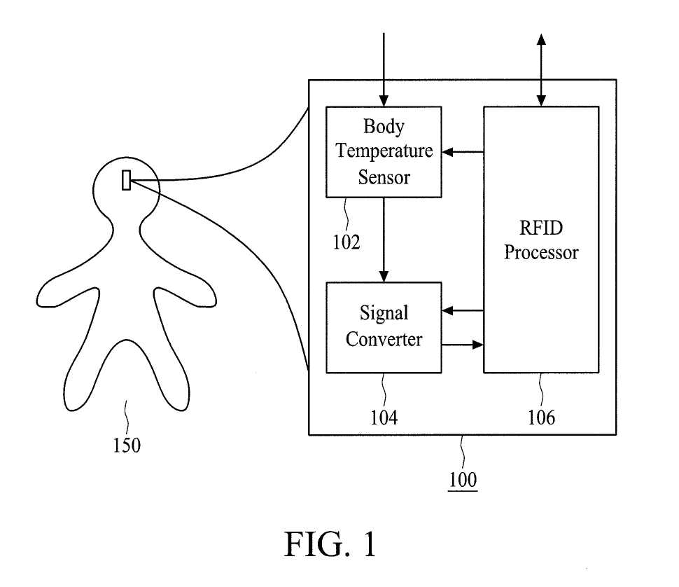

FIG. 1 shows a physiology condition detection device according to an embodiment of the present invention. As shown in FIG. 1, the physiology condition detection device 100 is attached to a human body 150, and comprises a body temperature sensor 102, a signal converter 104 and an RFID processor 106. The body temperature sensor 102 is configured to sense the temperature of the human body 150. The signal converter 104, which can be implemented by an analog-to-digital converter, is configured to convert the analog temperature signal provided by the body temperature sensor 102 to digitized temperature data. The RFID processor 106 is configured to control the operation of the body temperature sensor 102 and the signal converter 104, to report the digitized temperature data and to act as an interface with external devices.

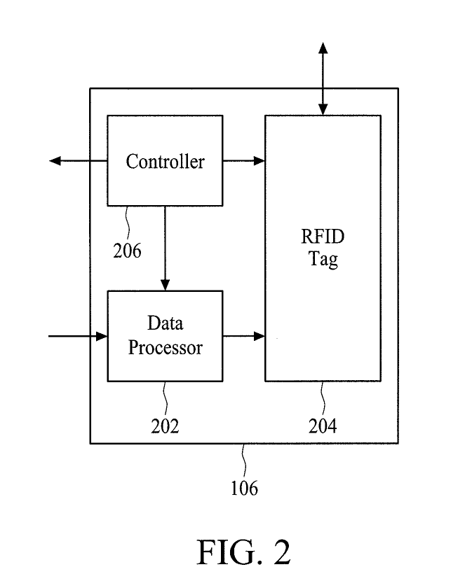

FIG. 2 shows the block diagram of the RFID processor 106 according to an embodiment of the present invention. As shown in FIG. 2, the RFID processor 106 comprises a data processor 202, an RFID tag 204 and a controller 206. The data processor 202 is configured to perform a data processing procedure on the digitized temperature data to generate report data. The RFID tag 204 is configured to report the report data and performs RFID communication with other devices. The controller 206 is configured to control the operation of the data processor 202, the RFID tag 204, the body temperature sensor 102 and the signal converter 104.

During a normal state, the physiology condition detection device 100 is idle, neither sensing nor receiving data. Therefore, the physiology condition detection device 100 consumes little power during the normal state. Upon receiving a control signal, the physiology condition detection device 100 is activated and enters a sensing state. Initially, the physiology condition detection device 100 is charged by an electromagnetic wave received by the RFID tag 204. Next, the body temperature sensor 102 performs a temperature sensing procedure, the signal converter 104 converts the analog temperature signal to digitized temperature data, and the data processor 202 performs a data processing procedure on the digitized temperature data to generate report data. Accordingly, the RFID tag 204 then reports the report data.

According to an embodiment of the present invention, the physiology condition detection device 100 does not comprise a transmitter. Accordingly, the RFID tag 204 is configured to report a first binary value, e.g. 1, when receiving a probing signal, and reports a second binary value, e.g. 0, when not receiving a probing signal.

According to another embodiment of the present invention, the data processor 202 is configured to collect the digitized temperature data. Next, the data processor 202 selects a peak value from the collected digitized temperature data as the report data. According to another embodiment of the present invention, the data processor 202 calculates an average value of the collected digitized temperature data as the report data. According to yet another embodiment of the present invention, the data processor 202 calculates a weighted average value of the collected digitized temperature data as the report data. That is, the data processor 202 provides different weights for different values of the collected digitized temperature data, and then calculates an average value of the weighted collected digitized temperature data.

It can be seen that since the physiology condition detection device 100 is idle during the normal state, and the physiology condition detection device 100 does not have a transmitter, the physiology condition detection device 100 consumes much less power than devices used in prior techniques. In addition, since the physiology condition detection device 100 is charged after being activated; that is, the physiology condition detection device 100 only consumes power when activated, wherein the power consumed in the sensing state is provided by the electromagnetic wave, and the physiology condition detection device 100 can function without batteries.

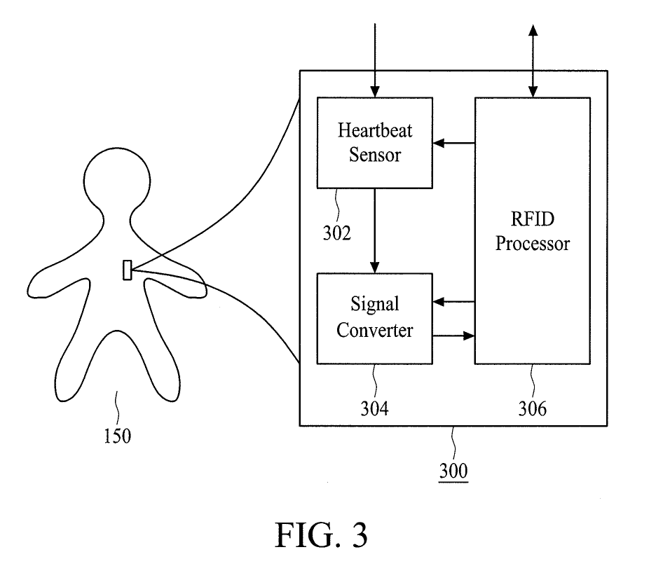

FIG. 3 shows a physiology condition detection device according to another embodiment of the present invention. As shown in FIG. 3, the physiology condition detection device 300 is attached to a human body 150, and comprises a heartbeat sensor 302, a signal converter 304 and an RFID processor 306. The heartbeat sensor 302 is configured to sense the heartbeat of the human body 150. The signal converter 304 is configured to convert the analog heartbeat signal provided by the heartbeat sensor 302 to digitized temperature data. The RFID processor 306 is configured to control the operation of the heartbeat sensor 302 and the signal converter 304, to report the digitized heartbeat data and to act as an interface with external devices. The heartbeat sensor 302 and body temperature sensor 102 are examples of physiology condition sensors, and heartbeat and body temperature are corresponding physiology conditions, respectively.

The operation of the physiology condition detection device 300 is similar to that of the physiology condition detection device 100 with the difference that the heartbeat sensor 302 senses a heartbeat of the human body 150 rather than the body temperature of the human body 150 as the body temperature sensor 102 does. According to the embodiments of the present invention, the physiology condition detection device 300 can also be used to sense the brain waves.

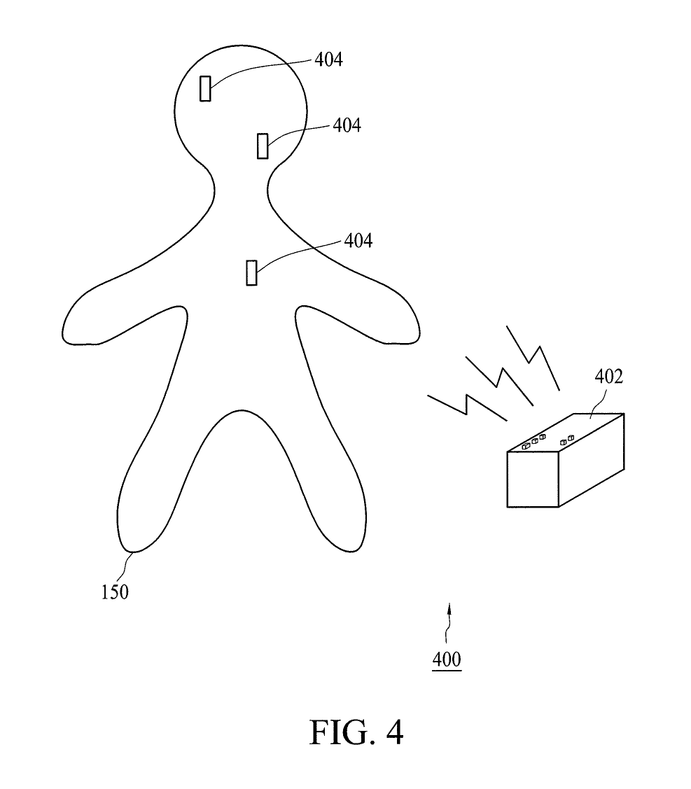

FIG. 4 shows a physiology condition detection system according to an embodiment of the present invention. As shown in FIG. 4, the physiology condition detection system 400 comprises an RFID reader 402 and a plurality of physiology condition detection devices 404. Each of the physiology condition detection devices 404 has a structure similar to that of the physiology condition detection device 100 or the physiology condition detection device 300. In addition, the plurality of physiology condition detection devices 404 are attached to different areas of the human body 150 and each performs its own sensing function. For example, one physiology condition detection device 404 may be attached to the forehead of the human body 150 and senses the body temperature of the human body 150. One physiology condition detection device 404 may be attached to the chest of the human body 150 and sense the heartbeat of the human body 150. One physiology condition detection device 404 may be attached to the temple of the human body 150 and senses the brain waves of the human body 150.

The RFID reader 402 is configured to broadcast control signals to the plurality of physiology condition detection devices 404, wherein some control signals may be broadcasted periodically, and some control signals may be broadcasted upon request. After a physiology condition detection device 404 receives the corresponding control signal and is thus activated, the RFID reader 402 then charges the specific physiology condition detection device 404 by transmitting an electromagnetic wave. After the specific physiology condition detection device 404 is charged and performs its own sensing function, the RFID reader 402 then transmits a series of probing signals to the specific physiology condition detection device 404. Next, the specific physiology condition detection device 404 reports the sensing results after receiving or not receiving each probing signal.

In conclusion, the physiology condition detection device and system provided by the present invention use RFID communication technique and do not require batteries or transmitters. Therefore, the physiology condition detection device and system provided by the present invention are capable of measuring physiology condition wirelessly and continuously.

Although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A physiology condition detection device, comprising:
a physiology condition sensor, configured to sense physiology condition;
a signal converter, configured to convert the sensed physiology condition to digitized physiology data; and
a radio frequency identification (RFID) processor, configured to control the physiology condition sensor and signal converter and to report the digitized physiology data.

2. The physiology condition detection device of claim 1, wherein the RFID processor is configured to report a first binary value when receiving a probing signal, and report a second binary value when not receiving a probing signal.

3. The physiology condition detection device of claim 1, wherein the RFID processor is configured to receive power provided by an electromagnetic wave so as to charge the physiology condition detection device.

4. The physiology condition detection device of claim 3, wherein the RFID processor is configured to report the digitized physiology data after being charged.

5. The physiology condition detection device of claim 1, wherein the physiology condition sensor is configured to sense body temperature.

6. The physiology condition detection device of claim 1, wherein the physiology condition sensor is configured to sense heartbeat condition.

7. The physiology condition detection device of claim 1, wherein the physiology condition sensor is configured to sense brain waves.

8. The physiology condition detection device of claim 1, wherein the RFID processor comprises:
a data processor, configured to collect the digitized physiology data and select a peak value from the collected digitized physiology data; and
an RFID tag, configured to report the peak value.

9. The physiology condition detection device of claim 1, wherein the RFID processor comprises:
a data processor, configured to collect the digitized physiology data and to provide an average value of the collected digitized physiology data;
an RFID tag, configured to report the average value.

10. The physiology condition detection device of claim 1, wherein the RFID processor comprises:
a data processor, configured to collect the digitized physiology data and to provide a weighted average value of the collected digitized physiology data;
an RFID tag, configured to report the weighted average value.

11. A physiology condition detection system, comprising:
a radio frequency identification (RFID) reader; and
at least a physiology condition detection device, configured to communicate with the RFID reader, wherein each physiology condition detection device comprises:
a physiology condition sensor, configured to sense physiology condition;
a signal converter, configured to convert the sensed physiology condition to digitized physiology data; and
an RFID processor, configured to control the physiology condition sensor and the signal converter and to report the digitized physiology data to the RFID reader.

12. The physiology condition detection system of claim 11, wherein the RFID reader is configured to broadcast a probing signal, and the RFID processor of the at least a physiology condition detection device is configured to report a first binary value when receiving a probing signal, and to report a second binary value when not receiving a probing signal.

13. The physiology condition detection system of claim 11, wherein the a physiology condition detection device.

14. RFID reader is configured to provide an electromagnetic wave to charge the at least The physiology condition detection system of claim 13, wherein the RFID reader is configured to broadcast a probing signal to the at least a physiology condition detection device after charging the at least a physiology condition detection device.

15. The physiology condition detection system of claim 14, wherein the RFID processor of the at least a physiology condition detection devices is configured to report a first binary value when receiving a probing signal, and to report a second binary value when not receiving a probing signal.

16. The physiology condition detection system of claim 11, wherein the physiology condition sensor of the at least a physiology condition detection devices is configured to sense body temperature, heartbeat condition, brain wave, or the combination thereof.

17. The physiology condition detection system of claim 11, wherein the RFID processor of the at least a physiology condition detection devices comprises:
a data processor, configured to collect the digitized physiology data and to select a peak value from the collected digitized physiology data; and
an RFID tag, configured to report the peak value to the RFID reader.

18. The physiology condition detection system of claim 11, wherein the RFID processor of the at least a physiology condition detection devices comprises:
a data processor, configured to collect the digitized physiology data and to provide an average value of the collected digitized physiology data;
an RFID tag, configured to report the average value.

19. The physiology condition detection system of claim 11, wherein the RFID processor of the at least a physiology condition detection devices comprises:
a data processor, configured to collect the digitized physiology data and to provide a weighted average value of the collected digitized physiology data; and
an RFID tag, configured to report the weighted average value.
